(19) United States
(12) Patent Application Publication
(10) Pub. No.: US 2012/0128602 A1
(43) Pub. Date: May 24, 2012

(54) SKIN COMPOSITIONS CONTAINING LOW LEVELS OF COLORANTS

(75) Inventors: Kelly Ann Dobos, Cincinnati, OH (US); Kevin Phifer, Cincinnati, OH (US); Alisa Ivory Smith, Cincinnati, OH (US); Jordan Cordeiro, Liberty Township, OH (US); Jessica Linn Rustici-Jones, Covington, KY (US); Sara Elizabeth Shirley, North Augusta, SC (US); Kelly Anne Kanellis, Fort Thomas, KY (US)

(73) Assignee: Kao Corporation, Tokyo (JP)

(21) Appl. No.: 13/298,660

(22) Filed: Nov. 17, 2011

Related U.S. Application Data
(60) Provisional application No. 61/415,642, filed on Nov. 19, 2010.

(51) Int. Cl. A61K 8/49; A61K 8/06; A61Q 1/02; A61Q 19/04; A61Q 19/08; A61Q 17/04; A61K 8/35; A61K 8/96

(52) U.S. Cl. 424/59; 424/63

(57) ABSTRACT

A composition for providing enhanced skin appearance, particularly in terms of providing the appearance of reduced dryness and flakiness for an extended period of time, is disclosed. The compositions include low levels of skin coloring agents, including self-tanning compounds, such as DHA and/or erythrulose, in a topical cosmetic base. The levels of self-tanning compounds utilized are lower than those typically found in self-tanning compositions. A method for enhancing skin appearance utilizing those compositions is also disclosed.

The present invention relates to and claims priority from U.S. Provisional Patent Application No. 61/415,642, Dobos et al, filed Nov. 19, 2010, and incorporated herein by reference.

### TECHNICAL FIELD

The present invention relates to topical compositions (for example, lotions, oils, mousses, sprays, serums, and gels) which enhance the appearance of skin (including dry, scaly skin) to which they are applied.

### BACKGROUND

The current approach to enhancing the appearance of skin generally relies on humectants, emollients, or occlusive agents. When applied to skin, these ingredients cause an immediate and generally temporary improvement in skin appearance by hydrating the stratum corneum, and smoothing down dry squames which, because of their size and whitish color, cause the skin to appear dry. These beneficial effects are easily undone when the skin is washed or bathed. It would be useful to have another, more effective, longer-lasting mechanism for dealing with dry, flaky, scaly skin appearance issues, and to provide the skin with an overall healthy appearance.

Colorant or self-tanning materials (such as dihydroxyacetone (DHA) and/or erythrulose) are known for use at relatively high levels (up to 15%) in self-tanner and instant bronzer products. These materials provide a tan-like coloration to the skin, utilizing a Maillard-like reaction, which can last for several (3-10) days. Unfortunately, if not used carefully, such high levels of colorants can cause unsightly streaks, spots, and unwanted deposits of color on hairs and nails.

U.S. Pat. No. 6,451,293, Schreier et al, issued Sep. 17, 2002, incorporated herein by reference, describes self-tanning compositions containing combinations of 1% DHA and 1% erythrulose. Decreased drying out of the skin is described as one of the benefits obtained. U.S. Pat. No. 5,626,839, Scales-Medeiras, issued May 6, 1997, incorporated by reference herein, describes a composition containing a self-tanning agent together with a fluorescent agent. The self-tanning agent is taught to be present at from about 0.05% to about 90% of the composition; the fluorescent agent is said to indicate the extent of even coverage by the self-tanning agent thereby preventing streaking and light/dark spots. U.S. Published Patent Application 2005/0100516, Dann, published May 12, 2005, incorporated herein by reference, describes self-tanning compositions containing a self-tanning agent (e.g., 1-25% DHA), a surfactant, and a hydro-alcohol carrier, which are said to provide and more even skin coverage than conventional compositions.

The present invention provides a new approach to enhancing skin appearance using very low levels of skin coloring agents, including self-tanning agents, such as dihydroxyacetone (DHA) and/or erythrulose, in conventional skin care topical formulations. DHA, erythrulose, select dyes, and other ingredients which react with proteins in the skin or otherwise stain the skin, can be used at ultra-low levels to provide a natural coloration to the squames and thus improve appearance. Because the colored compounds are bound to proteins, the effect is long-lasting and sustained until the stratum corneum completes its normal turnover process. Thus, these compositions of the present invention make skin appear more moisturized, healthier and more radiant over an extended period of time. These ultra-low levels also reduce concerns about streaking, spotting, and unwanted color on hands and nails associated with high levels of colorants.

### SUMMARY

The present invention relates to topical compositions which comprise skin coloring agents, such as from about 0.01% to about 0.9% of one or more mono- or poly-carbonyl self-tanning compounds, in a topical cosmetic base. Compositions can be substantially free from fluorescent dyes. Examples of self-tanning compounds include DHA, erythrulose, and mixtures of those materials.

The present invention also encompasses a method for enhancing skin appearance (e.g., treating dry and/or scaly skin) by applying to the skin an effective amount of a topical skin composition comprising a skin coloring agent, such as from about 0.01% to about 1.2% of one or more mono- or poly-carbonyl self-tanning compounds in a topical cosmetic base. Again, the compositions can be free from fluorescent dyes. Examples of self-tanning agents include DHA, erythrulose, and mixtures of those materials.

All percentages and ratios cited herein are “by weight” unless otherwise specified.

All references cited in this application are incorporated by reference herein.

### DESCRIPTION OF THE DRAWING

FIG. 1 is a graphical representation of the data from Example II.

### DETAILED DESCRIPTION

The compositions of the present invention comprise a skin coloring agent(s), such as one or more mono- or poly-carbonyl self-tanning compounds in a topical cosmetic base. The self-tanning compounds are present in the composition at from about 0.01% to about 1.2%, for example from about 0.01% to about 0.9%; from about 0.1% to about 0.9%; and from about 0.1% to about 0.75%, by weight. When applied to skin, the compositions make the skin appear more moisturized, healthier and more radiant over an extended period of time.

The self-tanner colorant component is generally a mono- or poly-carbonyl compound, such as isatin, alloxan, ninhydrin, glyceraldehyde, mesotartaric aldehyde, glutaraldehyde, dihydroxyacetone (DHA), erythrulose, melanin, mahakanni, 5,6-dihydroxy indole, and mixtures thereof. These self-tanning materials are well-known to those skilled in the art. They provide a skin browning effect utilizing a reaction similar to the Maillard reaction. For example, DHA reacts chemically with the amino acid groups, which are part of the protein-containing keratin layer on the skin surface. Various amino acids react differently to DHA, producing different tones of coloration ranging from yellow to brown. The resulting pigments are called melanoidins. These are similar in coloration to melanin, the natural substance in the deeper skin layers, which brown or “tan” from exposure to UV rays. Dihydroxyacetone, erythrulose, and mixtures of those materials are preferred for use in the compositions of the present invention. Self-tanning agents, including DHA and erythrulose, are disclosed in U.S. Published Patent Application 2005/0100516, Dann, published May 12, 2005; U.S. Pat. No. 5,626,839, Scales-Medeiros, issued May 6, 1997; and U.S. Pat. No. 6,451,293, Schreier et al, issued Sep. 17, 2002, all of which are incorporated herein by reference.

Although the self-tanning materials (mono- or polycarbonyl compounds) are preferred for use in the present invention, any material that provides a temporary tan shade coloration to the skin may be used. Examples of those materials include acid dyes, reducing sugars, self-tanning agents and skin stains.

Examples of acid dyes include Yellow No. 203 (D&C Yellow No. 10, color index (CI) given as CI47005), Orange No. 205 (D&C Orange No. 4 (CI 15510)), Red No. 227 (D&C Red No. 33 (CI 17200)), Violet No. 401 (Ext. D&C Violet No. 2 (CI 607301)), and Black No. 401 (CI 20470)). Combinations of these dyes can be used. For example, useful combinations include Orange 4+Yellow 10+External Violet 2; another useful combination is Orange 4+Red 33+External Violet 2; another useful combination is Orange 4+Red 33+External Violet 2; or a combination of Orange 4 and Red 3.

Organic carboxylic acids, such as citric acid, malic acid, lactic acid, glycolic acid, and combinations thereof, can be used together with the acid dyes, described above.

Examples of reducing sugars that can be used as skin coloration agents include glucose, xylose, fructose, reose, ribose, arabinose, allose, tallose, altrose, mannose, galactose, lactose, sucrose, erythrose, glyceraldehydes, raffinose, alpha glucosyl rutin, Vaccinium myrtillus, and combinations thereof

Examples of useful self-tanning agents include dihydroxyacetone, melanin, mahakanni, erythrulose, glyceraldehyde (glycerol aldehyde) and related alcohol-aldehydes, various indoles and imidazoles and their derivatives, 5,6-dihydroxy indole and certain of its derivatives, and combinations thereof.

Examples of skin stains include extract of a plant from the family of Compositae and Asteraceae (e.g., chicory); Menthe piperta; Melva silvestris; Cynara Scolymus; Thea sinensis; Juglans regia; Lawsonia inermis; Castanea vulgaris; Asorum europaeum; Leonurus cardiaca; Ballota foetida; Ocimum basilicum; Stachys officinalis; Brunella vulgaris; Calamintha officinalis; Thymus vulgaris; Rosmarinus officinalis; Humulus lupulus; Vaccinium myrtillus; Arctotaphylos uva-ursi; Calluna vulgaris; Artemisian abisinthium; Artemisia vulgaris; Artemisia abrotonum; Artemisia glacialis; Artimesia mutellina; Artemisia spicata; Chamamelum nobile; Fraxinus excelsior; Syringa vulgaris; Jasminium grandiflorum; Lythrum salicaria; Althaea officinalis; Hysopus officinalis; Origanum majorana; Salvia officinalis; Melissa officinalis; Melittis melissophylum; Lavandula officinalis; Quercus robur; Fagus silvatica; Nepta cataria; Origanum dictamus; Thymus serpyllum; Cichorium intybus L.; Gymnema sylvestre; and combinations of those materials.

Other materials which are known to be useful as skin coloring agents include caramels, malva extracts, hibiscus extracts, tyrosines, green teas, glyceraldehydes, ginsengs, erythruloses, ferric compounds, annattos, ultramarine pigments, beta-carotenes, carmins, chromium oxides, D&C water-soluble dyes, bismuth compounds, FD&C water-soluble dyes, copper powders, guanines, walnut extracts, iron oxides, micas, and combinations thereof. Hennas, caramels and mixtures of those materials are preferred.

The compositions of the present invention generally include from about 0.01% to about 20%, for example, from about 0.05% to about 10%, from about 0.05% to about 2.5%, or from about 0.1% to about 1.5%, of these skin coloring agents. When self-tanning materials are used, it is preferred that they be present at from about 0.01% to about 1.2%, such as from about 0.1% to about 0.9%, or from about 0.1% to about 0.75% of the composition by weight. The precise level used will depend on the chemical nature of the colorant, the mechanism by which it provides coloration, and whether it acts to build up color slowly over time and usage.

Skin penetration agents may be used in the present invention to help the skin coloring agent penetrate into the skin. Such agents are well-known to persons formulating topical compositions. Examples of such skin penetration agents include benzyl alcohol, 2-benzyloxyethanol, propylene carbonate, gamma-butyrolactone, N-methylpyrrolidone, propylene glycol, isopentyldiol, butylene glycol, ethanol, glycerin, polyethylene glycols, caprylyl pyrrolidone, lauryl pyrrolidone, dimethylisosorbide, benzyl oxyethanol, phenoxyethanol, ethoxydiglycol, pyrrolidinone, and mixtures thereof. When present, such materials are used at levels at from about 0.05% to about 50%, generally from about 0.05% to about 10% of the composition.

The compositions into which the self-tanner colorants are incorporated are conventional topical cosmetic compositions and are very well-known in the art. Examples of such compositions are described in U.S. Published Patent Application 2005/0169879, Glover et al, published Aug. 4, 2005; U.S. Published Patent Application 2004/0175403, Lukenbach et al, published Sep. 9, 2004; and U.S. Published Patent Application 2003/0031688, Ghosh et al, published Feb. 13, 2003, all of which are incorporated herein by reference. Conventional ingredients, such as moisturizers, humectants, vehicles, emulsifiers, surfactants, preservatives, fragrances, anti-aging components, proteins, rheology control agents, vitamins, botanicals, oils, and sunscreen agents, may be included at their conventional art-established usage levels. The compositions may be formulated as, for example, oils, sprays, ointments, liquids, serums, mousses, lotions, gels or wipes. The compositions must be compatible with the colorant component and the colorant should be dispersed or dissolved uniformly throughout the compositions. Examples of such compositions of the present invention, including the description of how to make those compositions, are set forth later in this application.

The compositions of the present invention frequently include both hydrophobic and hydrophilic components, and may be advantageously formulated as emulsions. When an emulsion is formulated, an emulsifier may be included in the composition. While emulsifiers are well-known in the art, those that are suitable for topical use on skin are most desirable. Examples of emulsifiers that may be used in the present invention can be found in the Handbook of Cosmetic and Personal Care Additives, 2^{nd} Edition(2002), published by Synapse Information Resources, which is incorporated herein by reference in its entirety. Preferred emulsifiers suitable for use in the present invention include isoceteth-20, dicetyl phosphate, ceteth-10-phosphate, sodium stearate, stearic acid, cetearyl alcohol, stearamidopropyldimethylamine, behentrimonium methosulfate, sodium cocoyl taurate, cetearyl glucoside, sodium methyl oleoyl taurate, sodium lauryl sulfate, and the like, alone or in combination, with cetearyl glucoside being preferred.

Preferred emulsifiers, and particularly the glucosides, are believed to assist in enhancing the retention of water within the skin. For example, cetearyl glucoside is thought to strengthen the lipid structure within the skin, establishing a barrier to moisture loss.

The amount of emulsifier that may be included in the compositions of the present invention may vary, depending on the degree to which the properties provided by this component are desired in those compositions, but, if present, is generally included in an amount of from about 0.001% to about 10% by weight of the composition.

The compositions of the present invention may include surfactants. Anionic, nonionic, cationic, and amphoteric surfactants, and combinations thereof may be used. Suitable nonionic surfactants include, but are not limited to, alkyl glycosides, such as octoyl glucoside, decyl glucoside, and lauryl glucoside; ethylene oxide/propylene oxide copolymers; polyoxyethylene derivatives of polyol esters; and sucrose esters. When utilized, the amount of nonionic surfactant in the compositions of the present invention may range from about 0.1% to about 20%, for example from about 0.5% to about 10%, based on the total weight of the composition.

Betaine surfactants may also be useful in the compositions of the present invention. As used herein, betaines are derived from alkyl amidopropyl dimethylamine. They can exist in only two forms: cationic at low pH and isoelectric at intermediate pH. Suitable betaine surfactants include, but are not limited to, alkyl betaines, amidoalkyl betaines, phosphorobetaines, pyrophosphobetaines, and mixtures thereof. When utilized, the amount of betaine surfactant in the compositions of the present invention may range from about 0.1% to about 20%, for example from about 0.5% to about 10% by weight, based on the total weight of the composition.

Amphoteric surfactants may be useful in the compositions of the present invention. As used herein, amphoteric surfactants are derivatives of alkyl hydroxyethyl imidazolines formed through a reaction with sodium chloroacetates. They are true amphoterics in that they exist in three different forms depending on the pH: cationic at low pH, zwitteronic at intermediate pH and anionic at high pH. Suitable amphoteric surfactants include, but are not limited to, amphocarboxylates, amidosultaines, amphophosphates, carboxyalkyl alkyl polyamines, and mixtures of those materials. When utilized, the amount of amphoteric surfactant in the compositions in the present invention may range from about 0.1% to about 20%, for example from about 0.5% to about 10% by weight, based on the total weight of the composition.

Anionic surfactants may also be utilized in the compositions of the present invention. Suitable anionic surfactants include, but are not limited to, sodium lauryl sulfate, sodium laureth sulfate, sodium salt of fatty acids, cetyl phosphate taurates, acylamino acids, and lactylates. When utilized, the amount of anionic surfactant in the compositions of the present invention may range from about 0.1% to about 20%, for example from about 0.5% to about 10%, by weight of the total composition.

The beneficial effects provided by the compositions and methods of the present invention can be formulated in a number of different cosmetic forms. For example, the compositions of the present invention may be formulated in a variety of topical products, for example, facial moisturizers, eye creams and lotions, hand lotions, body lotions, and the like. They can also be incorporated onto wipes which are used to apply the composition to the skin surface. These compositions can be formulated into any suitable form, for example, a gel, a lotion, a cream, an ointment, a solution, a spray, or the like, as will be appreciated by the ordinary skilled artisan.

In some aspects of the present invention, for example, cream and lotion formulations, the composition may be provided in the form of a water-in-oil emulsion or an oil-in-water emulsion. As is well-known, in a water-in-oil emulsion, the oil phase is the continuous (or external) phase, and dispersed within the oil phase is the aqueous (or internal) phase. An oil-in-water emulsion is the opposite: the aqueous phase is the continuous phase and the oil phase is dispersed within the aqueous phase. Emulsion formulations can assist in providing uniform application of the composition onto the skin and possess good skin feel.

Even if the composition is not in the form of an emulsion, water and/or oils can be included in the composition. If water and/or oil are included, in an emulsion or otherwise, they can be included in any amount suitable to provide the desired properties in the finished composition. For example, if water is included in an emulsion or other composition (e.g., dispersion) form, it desirably may be present in an amount of from about 20% to about 95% by weight of the composition. If oil is present, it desirably may be present in an amount of from about 10% to about 95% by weight of the composition.

If present, the oil may be any of a number of components suitable for inclusion in a topical cosmetic composition. Illustrative oils, some of which may function as emollients, include silicone oils, triglyceride esters, natural oils, waxes, hydrocarbons, phospholipids, polyhydric and fatty alcohols, polyether derivatives, and they like, as well as those described in U.S. Pat. No. 5,980,921, Biedermann et al, issued Nov. 9, 1999, and U.S. Pat. No. 6,042,815, Kellner et al, issued Mar. 28, 2000, both incorporated herein by reference. Combinations of these oil materials may also be included in the present invention.

Illustrative of useful oils are silicone oils. Silicone oils may be provided in the form of one or more volatile silicones, non-volatile silicones, and mixtures thereof. Exemplary silicone oils include, for example, cyclomethicone, phenyl trimethicone, alkyl dimethicone, fluorinated silicones, dimethicone, and the like, or combinations thereof. It is thought that these oils can assist in enhancing uniform delivery and ease in spreading of the compositions of the present invention.

Although not essential, the compositions of the present invention may also include one or more optional ingredients typically used in topical compositions, such as, for example, thickeners, preservatives, emollients, moisturizing additives, neutralizers, fragrances, antioxidants, skin conditioners, or the like.

If included, any conventional cosmetic thickener may be utilized in the present invention. By way of example, the thickener can be in the form of a gum, cellulosic, acrylic polymer, carbomer, and the like, or combinations thereof. Especially suitable thickeners include, but are not limited to glyceryl stearate, cetyl alcohol, carbomer, C₁₀-C₃₀ alkylacrylate cross-polymer, and the like, or combinations thereof. The thickener may be provided in any suitable amount as would be understood by one skilled in the cosmetic formulation art. For example, the thickener may be present in an amount of from about 0.01% to about 5% by weight of the composition.

The composition optionally may further include a preservative. For example, the preservative may be selected to kill bacteria that might otherwise be sustained or multiplied in the composition. Preservatives suitable for use in cosmetic formulations are well-known to those skilled in the art. In this respect, the type of preservative chosen will depend upon the components and the structure of the composition. For example, some preservatives are selected to combat microorganisms that are sustained in water, while others are selected to combat microorganisms that are sustained in oil. Illustrative of suitable preservatives include ethylparaben, propylparaben, methylparaben, EDTA or salts thereof (such as disodium EDTA), phenoxyethanol, DMDM hydantoin, and the like, or combinations thereof. The preservative may be present in any effective amount, such as an amount of from about 0.01% to about 3% by weight of the composition.

The composition may also include an emollient. As used herein, emollient refers to a material used for the prevention or relief of skin dryness, as well as for the protection of the skin. The emollient desirably may be selected to compliment the emollient properties of the oil mentioned previously, if the latter is present in the composition. Any suitable emollient may be included, as long as it is compatible with the self-tanning colorant, and a wide variety of such emollients are known and may be used. Sagarin, Cosmetics, Science and Technology, 2^{nd} Edition, Volume 1, pgs. 32-43 (1972), incorporated herein by reference, contains numerous examples of materials suitable for use as an emollient. By way of example, the emollient can be selected from C₁₂-C₁₅ alkylbenzoate, C₁₂-C₁₅ alkyloctanoate, caprylic triglyceride, capric triglyceride, mineral oil, cetearyl alcohol, dimethicone, cyclomethicone, shea butter and the like, or combinations thereof. If present, the emollient may be included in any suitable amount, such as, for example, in an amount of from about 0.1% to about 10%, by weight of the composition.

The compositions of the present invention may also include one or more moisturizing additives. Any suitable moisturizing additive may be used. Illustrative moisturizing additives include, but are not limited to, esters, humectants, urea, glycerine, retinyl palmitate, petrolatum, gelatin, keratin amino acids, lysine, hydroxypropyl trimonium chloride, propylene glycol, natural botanical extracts, such as chamomile recutita extract, sambucus nigra extract, primula veris extract, helianthus annuus extract, and the like, as well as phospholipids, silicones, occulusive agents, natural oils, barbadensis gel, and the like, or combinations thereof. If included, the moisturizing additives may be included in any suitable amount, for example, in an amount of from about 0.0001% to about 25%, or from about 1% to about 10%, by weight of the composition.

If desired, the composition may further include one or more neutralizers, such as, for example, strong and weak bases. Any suitable neutralizer can be selected, as will be appreciated by one of ordinary skill in the art. Exemplary neutralizers suitable for use in the compositions of the present invention include sodium hydroxide, potassium hydroxide, ammonium hydroxide, diethanolamine, triethanolamine, 2-dimethylamino-2-methyl-1-propanol (DAMP), 2-amino-methyl-1-propanol (aminomethylpropanol) (AMP), and the like, or combinations thereof. Weak acids, such as citric acid or malic acid, may also be used. The neutralizer, if present, may be provided in any amount, for example, in an amount sufficient to achieve a desired pH for the composition. In this respect, the composition preferably has a pH of from about 4 to about 9, for example, from about 5 to about 8, and more typically from about 4.5 to about 7. Typically, the neutralizer is present in an amount of from about 0.01% to about 10% by weight of the composition, when used.

The composition may also include a fragrance, if desired. The fragrance may be selected from those most suitable for cosmetic preparations, as is well-known to those skilled in the cosmetic formulation arts. If included, the fragrance may be present in an amount of from about 0.01% to about 3% by weight of the composition.

An antioxidant may also be included in the compositions of the present invention, if desired. The antioxidant may be any antioxidant suitable for cosmetic preparations, as would be appreciated by one skilled the cosmetic formulation arts. Illustrative of suitable antioxidants include coenzyme Q10, vitamin C, vitamin E, superoxide dismutase (SOD), tocopheryl acetate, ascorbic acid, and the like, or combinations thereof. While the antioxidant may be included in any suitable amount, it may desirably be included in an amount of from about 0.00001% to about 5%, for example, from about 0.05% to about 1%, by weight of the composition.

The composition may further include, if desired, a skin conditioner. The skin conditioner may be any suitable skin conditioner, as will be appreciated those skilled in cosmetic formulation arts. Exemplary skin conditioners include phytantriol, panthenyl ethyl ether, primula varies extract, chamomi, sambucus nigra flower extract, panthenol, polyquaternium-51, cetyl alcohol, glycolic acid, stearyl alcohol, and the like, or combinations thereof. The skin conditioner can be included in the present compositions in any suitable amount, but may desirably be included in an amount of from about 0.1% to about 5% by weight of said composition, if used.

The compositions of the present invention are made using conventional cosmetic formulational techniques which would be well-known to those skilled in the cosmetic formulational arts. The precise steps will depend on the type of composition formulated and the specific components included.

The present invention also encompasses a method for enhancing skin appearance. In this method, an effective amount of a topical skin composition comprising a skin coloring agent, such as from about 0.1% to about 1.2% of one or more mono- or poly-carbonyl self-tanning compounds, in a topical cosmetic base is applied to the skin. For example, compositions may utilize DHA or erythrulose or mixtures thereof as the self-tanning compound. Further, compositions can be substantially free of fluorescent dyes (i.e., the compositions contain less than 0.05%, preferably less than 0.005%, of a fluorescent dye and can contain no fluorescent dye). As used herein, the phrase “effective amount” is the amount of the composition necessary to provide the skin appearance benefits of the present invention but not so much as to overuse or waste the composition or to provide an undesirable skin feel. Typically, the compositions of the present invention are applied in an amount of from about 0.25 to about 5 ml of composition per square centimeter of skin.

The present invention also encompasses the method for enhancing skin appearance wherein a topical skin composition comprising from about 0.01% to about 20% (for example, from about 0.05% to about 10%, or from about 0.05% to about 2.5%, or from about 0.1% to about 1.5%) of a skin coloring agent selected from acid dyes, reducing sugars, skin stains, and mixtures thereof, is applied to the skin in an effective amount.

The composition can be applied to skin (including, but not limited to, dry scaly skin and normal skin) in order to provide a moisturized, healthier and more radiant appearance.

### EXAMPLE I

A composition in the form of a lotion having the components and composition described in the table below and produced using conventional techniques, is formulated. This composition, when applied to the skin, results in enhanced skin appearance (healthier looking, more radiant, reduced appearance of dryness and flakiness) and moisturization to the skin, without resulting in any greasy feel.

| | RM in Finished |
|---|---|
| Ingredient List | Product % wt/wt |
| Water | 76.5312 |
| Hydroxyethyl Acrylate/Sodium Acryloyldimethyl | 0.5000 |
| Taurate Copolymer (and) Isohezecane (and) | |
| Polysorbate 60 | |
| Glycerin | 8.5000 |
| Glyceryl Stearate | 2.2000 |
| Cetearth-20 | 0.7200 |
| PEG-100 Stearate | 0.7200 |
| Cetearyl Alcohol | 3.3000 |
| Stearic Acid | 0.5000 |
| Glycine Soja (Soybean Oil) & Hydrogenated | 2.5000 |
| Cottonseed Oil | |
| Simmondsia Chinensis (Jojoba) Seed Oil | 0.5000 |
| Helianthus Annuus (Sunflower) Seed Oil | 0.7500 |
| Caprylic/Capric Triglyceride | 0.7500 |
| BHT | 0.1000 |
| Citric Acid | 0.0030 |
| Dihydroxyacetone | 0.7000 |
| Polymide-1 | 0.0100 |
| Fragrance | 0.2500 |
| DMDM Hydantoin | 0.6000 |
| Phenoxyethanol (and) Ethylhexylglycerin | 0.8500 |
| Caramel | 0.0158 |
| | 100.0000 |

### EXAMPLE II

A test to measure the skin coloration capacity of the compositions of the present invention and the retention of that coloration after usage has been stopped, was carried out.

A product of the present invention, having the following formulation, was used. The product was made by conventional processes.

Composition

| | RM in |
|---|---|
| | Finished |
| | Product |
| Ingredient List | % wt/wt |
| Water | 68.0010% |
| Propylparaben NF | 0.1000% |
| Methylparaben NF | 0.2000% |
| Hydroxyethyl Acrylate/Sodium Acryloyldimethyl Taurate | 1.6000% |
| Copolymer (and) Isohexadecane (and) Polysorbate 60 | |
| Glycerin | 8.0000% |
| Dipropylene Glycol | 5.0000% |
| Ethoxydiglycol | 1.5000% |
| Xanthan Gum | 0.1000% |
| Glyceryl Stearate (and) PEG-100 Stearate | 0.7500% |
| Cetearyl Alcohol | 3.5000% |
| Behenyl Alcohol | 0.2500% |
| Ceteareth-20 | 1.2500% |
| Petrolatum | 2.0000% |
| Octyldodecyl Myristate | 0.5000% |
| Isopropyl Isostearate | 1.2500% |
| Ethylhexyl Isononanoate | 0.5000% |
| Mineral Oil | 1.7000% |
| Steareth-2 | 0.2500% |
| Glyceryl Stearate | 0.5000% |
| BHT | 0.0200% |
| Aluminum Starch Octenylsuccinate | 0.5000% |
| Dimethicone | 1.7500% |
| Citric Acid | 0.0280% |
| Sodium Citrate | 0.0010% |
| Dihydroxyacetone | 0.3500% |
| DMDM Hydantoin (55%) | 0.4000% |
| | 100.000% |

Four groups of participants were tested:

Group 1—African-American (12 participants)

Group 2—Caucasian (fair-medium complexion) (12 participants)

Group 3—Caucasian (medium-tan complexion) (12 participants)

Group 4—Hispanic (8 participants)

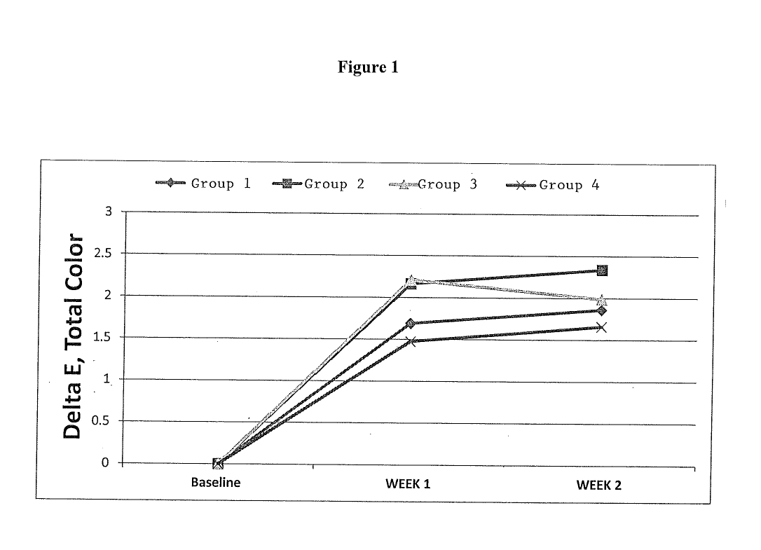

The product was applied ad lib by the panelists (approximately 2mg/cm² per application). A Minolta Chromameter (CR300) instrument was used to take color measurements on eight skin sites per panelist at each time point: baseline, week 1 of usage, week 2 of usage, 1 week post-usage. A single Chromameter measurement (L*a*b*, single reading in triplicate) was conducted on the leg calf of each panelist. L*a*b* values were then reported as change from baseline and Delta E (ΔE) for each panelist and an average for all panelists taken. The results are reported in the following table and in FIG. 1.

| Group | Ethnicity | Time | ΔL* | Δa* | Δb* | ΔE |
|---|---|---|---|---|---|---|
| 1 | African- | 2 weeks of usage | 0.36 | 0.14 | 0.53 | 1.60 |
| | American | | | | | |
| 2 | Caucasian- | 2 weeks of usage | 0.22 | −0.21 | 0.94 | 2.02 |
| | F-M | | | | | |
| 3 | Caucasian- | 2 weeks of usage | 0.67 | −0.31 | 1.09 | 2.08 |
| | M-T | | | | | |
| 4 | Hispanic | 2 weeks of usage | 0.48 | −0.04 | 0.51 | 1.65 |
| 1 | African- | 1 week post-usage | −0.42 | 0.37 | 1.13 | 1.86 |
| | American | | | | | |
| 2 | Caucasian- | 1 week post-usage | −0.27 | −0.06 | 1.28 | 2.34 |
| | F-M | | | | | |
| 3 | Caucasian- | 1 week post-usage | −0.02 | 0.15 | 1.38 | 1.99 |
| | M-T | | | | | |
| 4 | Hispanic | 1 week post-usage | 0.07 | 0.16 | 0.77 | 1.66 |

These data show a light coloration of the skin during the first week of usage, providing a look of moist and healthy skin, and a maintenance of that light coloration over the second week of usage. The second set of readings in the table (comparison of ΔE values, first set vs. corresponding group in second set) shows good retention of color after usage has stopped.

What is claimed is:

1. A composition which comprises from about 0.01% to about 0.9% of one or more mono- or poly-carbonyl self-tanning compounds in a topical cosmetic base.

2. The composition of claim 1 which is substantially free of fluorescent dyes.

3. The composition of claim 2 wherein the self-tanning compounds are selected from isatin, alloxan, ninhydrin, glucoaldehydes, mesotartaric aldehydes, glutaraldehyde, dihydroxyacetone, erythrulose, melanin, mahakanni, 5,6-dihydroxy indole, and mixtures thereof.

4. The composition according to claim 3 wherein the self-tanning compound is selected from DHA, erythrulose, and mixtures thereof

5. The composition according to claim 4 which includes from about 0.1% to about 0.75% of the self-tanning compound.

6. The composition according to claim 4 in the form of an emulsion.

7. The composition according to claim 4 formulated in a form selected from oils, sprays, ointments, liquids, serums, mousses, lotions, gels and wipes.

8. The composition according to claim 4 wherein the topical cosmetic base comprises one or more ingredients selected from moisturizers, humectants, vehicles, emulsifiers, surfactants, preservatives, fragrances, anti-aging components, proteins, rheology control agents, vitamins, botanicals, oils and sunscreen agents.

9. The composition according to claim 4 which additionally includes a skin coloring agent selected from acid dyes, reducing sugars, skin stains, and mixtures thereof

10. A method for enhancing skin appearance by applying to the skin an effective amount of a topical skin composition comprising from about 0.01% to about 1.2% of one or more mono- or poly-carbonyl self-tanning compounds in a topical cosmetic base.

11. The method according to claim 10 wherein the composition is substantially free of fluorescent dyes.

12. The method according to claim 11 wherein the self-tanning compound is selected from isatin, alloxan, ninhydrin, glucoaldehydes, mesotartaric aldehydes, glutaraldehyde, dihydroxyacetone, erythrulose, melanin, mahakanni, 5,6-dihydroxy indole, and mixtures thereof.

13. The method according to claim 11 wherein the self-tanning compound is selected from DHA, erythrulose, and mixtures thereof

14. The method according to claim 13 wherein the composition includes from about 0.1% to about 0.9% of the self-tanning compound.

15. The method according to claim 14 wherein the composition includes from about 0.1% to about 0.75% of the self-tanning compound.

16. The method according to claim 13 wherein the composition is in the form of an emulsion.

17. The method according to claim 13 wherein the composition is formulated in a form selected from oils, ointments, sprays, liquids, serums, mousses, lotions, gels, and wipes.

18. The method according to claim 13 wherein the topical cosmetic base comprises one or more ingredients selected from moisturizers, humectants, vehicles, emulsifiers, surfactants, preservatives, fragrances, anti-aging components, proteins, rheology control agents, vitamins, botanicals, oils and sunscreen agents.

19. The method according to claim 13 wherein the composition is applied to the skin in an amount of from about 0.25 to 5 ml per square centimeter of skin.

20. The method according to claim 13 wherein the composition additionally includes a skin coloring agent selected from acid dyes, reducing sugars, skin stains, and mixtures thereof.

21. A composition which comprises from about 0.1% to about 20% of one or more skin coloring agents in a topical cosmetic base.

22. The composition according to claim 21 where the skin coloring agent is selected from acid dyes, reducing sugars, skin stains, and mixtures thereof

23. The composition according to claim 22 wherein the skin coloring agent comprises from about 0.1% to about 1.5% of the composition.

24. The composition according to claim 23 formulated in a form selected from oils, sprays, ointments, liquids, serums, mousses, lotions, gels and wipes.

25. The composition according to claim 23 wherein the topical cosmetic base comprises one or more ingredients selected from moisturizers, humectants, vehicles, emulsifiers, surfactants, preservatives, fragrances, anti-aging components, proteins, rheology control agents, vitamins, botanicals, oils and sunscreen agents.

26. A method for enhancing skin appearance by applying to the skin an effective amount of a topical skin composition comprising from about 0.01% to about 20% of one or more skin coloring agents in a topical cosmetic base.

27. A method according to claim 26 wherein the skin coloring agent is selected from acid dyes, reducing sugars, skin stains, and mixtures thereof.

28. The method according to claim 27 wherein the skin coloring agent comprises from about 0.1% to about 1.5% of the composition.
